(19) United States
(12) Patent Application Publication
(10) Pub. No.: US 2026/0083585 A1
(43) Pub. Date: Mar. 26, 2026

(54) ORAL USE OF AN IN-MOUTH WEARABLE DEVICE IN THE TREATMENT OF DYSPHAGIA

(71) Applicants: TONGUE LAB EUROPE LTD, London (GB); NATIONAL UNIVERSITY CORPORATION TOKYO MEDICAL AND DENTAL UNIVERSITY, Tokyo (JP)

(72) Inventors: Ryosuke Yanagida, Tokyo (JP); Haruka Tohara, Tokyo (JP); Jean-Michel Mauclaire, Paris (FR); Koji Hara, Yokosuka (JP)

(73) Assignees: TONGUE LAB EUROPE LTD, London (GB); NATIONAL UNIVERSITY CORPORATION TOKYO MEDICAL AND DENTAL UNIVERSITY, Tokyo (JP)

(21) Appl. No.: 19/108,416

(22) PCT Filed: Aug. 31, 2023
(86) PCT No.: PCT/IB2023/000524

§ 371 Date: Mar. 3, 2025

Related U.S. Application Data
(60) Provisional application No. 63/374,268, filed on Sep. 1, 2022.

(51) Int. Cl. A61F 5/56

(52) U.S. Cl. CPC A61F 5/566

(57) ABSTRACT

Disclosed is a method for treating dysphagia comprising: fitting a patient afflicted with dysphagia with a TRP oral retainer-based device having an arch for limiting movement of the patient's tongue, the device to be worn by the patient at night starting at about bedtime and during sleep, preferably for at least 8 hours; providing instructions to the patient to wear the device nightly for a period of time from about 2 to about 18 months (the wearing period) as indicated; monitoring the patient's tongue pressure at one or more time points during the wearing period, wherein an increase in tongue strength, as measured for example by tongue pressure (TP) compared to baseline or compared to a prior monitoring time point during the wearing period, indicates that the patient receives a benefit from wearing the device. An increase in tongue strength has been shown to correlate closely with reducing oropharyngeal dysphagia and its attendant symptoms and pathologies.

### FIELD OF THE DISCLOSURE

The present disclosure relates to a method of treating dysphagia, especially oropharyngeal dysphagia.

### BACKGROUND OF THE DISCLOSURE

### Dysphagia

Aging itself and many aging-associated degenerative diseases may induce dysphagia [1]. Because dysphagia causes dehydration, malnutrition, asphyxia, and aspiration pneumonia, it is one of the most life-threatening factors for the elderly [2, 3].

Feeding and swallowing can be divided into five stages: preoral (anticipatory), preparatory, oral, pharyngeal, and esophageal [4]. In these processes the tongue plays an important role, assisting in mastication, gustatory sensation, inducing salivation, bolus formation, and propelling the bolus into the pharynx. A significant amount of tongue strength (tongue pressure) is believed to be necessary for the preparatory and oral stages to mix food and saliva into a bolus and pass it to the pharynx [5]. There is evidence that dysphagia occurs due to a reduction in the ability of the tongue to propel the bolus into the pharynx. Thus, evaluating tongue strength is an important factor in the rehabilitation of feeding and swallowing. [15]

The tongue plays an essential role in mastication and deglutition, cooperating with the lips, mandible, pharynx, and larynx. Therefore, tongue function impairment negatively influences masticatory and swallowing movements, which can lead to dysphagia, difficulty swallowing [42]. Dysphagia is more common among older adults. In turn, it can lead to choking or aspiration of food into the airway. Both are potentially life-threatening with aspiration often leading to serious and potentially lethal airway and pulmonary infections].

Quantitative evaluation of tongue function is essential for dysphagia rehabilitation, and tongue pressure (TP) is used as a quantitative and convenient index of tongue strength. Decreased TP is associated with reduced swallowing and masticatory function [1, 2]; TP can be decreased by aging as well as pathological conditions that have been associated with dysphagia, such as cerebrovascular diseases [3], neuromuscular dysfunction [4,], Parkinson's disease [5], and sarcopenia [6, 41]. Therefore, a reduction in TP is a valuable index for rehabilitation in dysphagia cases, particularly oropharyngeal dysphagia [15,40]. Regarding the relationship between clinical symptoms and TP, prolonged mealtime, decrease in meal amount [7], the occurrence of aspiration [8], and pharyngeal residue [9] have been associated with decreased TP. Consequently, it is necessary to improve TP during rehabilitation. Conventional rehabilitation regimens include exercises such as balloon tongue resistance training [10], tongue protrusion training [11], and tongue pressure resistance training [12]. But dysphagia patient willingness and/or ability to perform exercises, especially on a sustained basis is often weak or absent and consequently patient compliance has been low.

Decreased tongue pressure (TP) levels have been associated with impaired swallowing and pathologies associated therewith such as dysphagia. The tongue right positioner (TRP) device has been used as an oral device to treat conditions such as obstructive sleep apnea (OSA), snoring, tongue dysfunctions, malocclusion, and labioversion. The effects of TRP on OSA may be associated with improved peroral muscle function. However, the device's effectiveness in addressing inadequate swallowing functions and particularly those associated with dysphagia has not been adequately investigated and remains unclear. Accordingly, it is an aim of the present invention to investigate the effects of the TRP on tongue strength and function in patients with dysphagia and to use the TRP to increase tongue strength in such patients, thereby alleviating difficulty in swallowing and reducing the risk of aspiration.

### The TRP Device and its Prior Uses

The TRP device has been previously used to treat sleep apnea and snoring in addition to being used for tongue and oral cavity remodeling. It has also been used to treat bruxism and some orthodontic problems, such as malocclusion. Some suitable embodiments of the TRP device are described in International (PCT) Patent Application PCT/IB2017/054130 published as WO/2018/008002. Earlier versions of the device have been described in patent applications published as WO/2010/015685 and WO/2012/085672 and in principle such devices can be used as well. The particular device used in the studies that gave rise to this disclosure is further described below.

Prior to the study disclosed here, the TRP was not used to treat dysphagia. Moreover, the patient populations of the prior uses for TRP are distinct from the dysphagia population. For example, dysphagia is not associated with children or teenagers. Additionally, even older patients, who might suffer from apnea do not necessarily also have dysphagia.

### BRIEF DESCRIPTION OF THE DRAWINGS

FIGS. 1A-1C are photographs of examples of tongue right positioners such as those employed in the studies detailed in this disclosure. FIG. 1A depicts an occlusal view taken from the bottom up of a rearward arch device: FIG. 1B depicts an occlusal view taken from the bottom up of a forward arch device. FIG. 1C depicts a perspective view of a forward-arched device as in FIG. 1B

FIG. 2 is a series of plots of tongue pressure between baseline and follow-up values for each study participant. Each number in a circle indicates an assigned number for each participant as further shown in Table 1.

FIG. 3 is a series of box plots showing results of the study. The upper left panel is a box plot of TP. The bottom of the box is the starting value and the top is the value at evaluation. The x marks the mean value and the horizontal bar marks the median value.

### SUMMARY OF THE DISCLOSURE

Disclosed is a method for treating oropharyngeal dysphagia comprising: fitting a patient afflicted with dysphagia with a TRP oral retainer-based device having an arch for limiting movement of the patient's tongue, the device to be worn by the patient at night starting at about bedtime and during sleep, preferably for at least 8 hours; providing instructions to the patent to wear the device nightly for a period of time from about 2 to about 18 months (the wearing period) as indicated; monitoring the patient's tongue strength at one or more time points during the wearing period, wherein an increase in tongue strength, as measured for example by tongue pressure (TP) compared to baseline or compared to a prior monitoring time point during the wearing period indicates that the patient receives a benefit from wearing the device. Wearing the device may be discontinued once a desired TP is achieved or wear can be continued, even indefinitely, or resumed if needed in order to substantially retain the gained benefit in tongue strength or even improve the gained benefit in tongue strength. The device may be optionally adjusted during the wearing period by replacement of the arch with an arch having different geometry and/or arch orientation as may be needed. Alternatively, the entire device can be replaced by another device having different orientation and/or geometry. Suitable devices are exemplified in FIGS. 8A to 8C, 10A to 10C and 11A to 11C of International PCT Patent Publication WO/2018/008002 reproduced below along with a description thereof. Indeed, if the tongue strength does not improve at the end of an initial device-wearing period. e.g. one to three months, the TRP can be adjusted or replaced. The new TRP arch or the arch of the new TRP may have a different length and/or width and/or angle with respect to the occlusal plane of the patient and/or a different orientation (backward or forward).

More specific embodiments included in the present disclosure include the following without limitation:

A method for treating dysphagia comprising:
fitting a patient afflicted with dysphagia with a TRP oral retainer-based device for wearing at night starting at about bedtime and during sleep, preferably for at least 8 hours;
providing instructions to the patient to wear the device nightly for a period of time from about 2 to about 18 months or longer (the wearing period) as indicated based on strength of the patient's tongue; monitoring the patient's tongue pressure at one or more time points during the wearing period.
wherein an increase in tongue strength compared to baseline, or compared to a prior monitoring time point during the wearing period, indicates that the patient receives a benefit from wearing the device or continues to receive a benefit from wearing the device and the patient's dysphagia is thereby treated; and
wherein the TRP device is for limiting movement of a posterior or anterior zone of the tongue and stimulating the tongue, while allowing respectively an anterior or posterior zone and lateral edges of the tongue to perform movements necessary for speech and swallowing and comprises:
   an attachment mechanism adapted to releasably attach the TRP device teeth of the patient, the attachment mechanism comprising
      a first surface adapted to be positioned on a vestibular side of upper teeth of the dental arcade of the patient and
      a pair of second surfaces, comprising a left second surface and a right second surface, each second surface in opposition to the first surface and adapted to be seated along at least one and up to four posterior teeth of the wearer on the lingual side of the posterior teeth and
      a connector for joining a left end of the first surface with the left second surface and a connector for joining a right end of the first surface with the right second surface
   a tongue-constraining mechanism having
      a first and second end wherein each end of the constraining mechanism is optionally removably secured to a corresponding second surface of the attachment mechanism and
      a component transverse to and spanning between the first and second surfaces and having a forward or rearward orientation, the component being configured to sit at the occlusal plane of the patient or at an angle thereto the angle being within the range from +0 to −30 degrees lower than the occlusal plane and to limit movement of the corresponding zone of the tongue thereby confining the range of motion of the tongue to a more limited three-dimensional space within the mouth of the patient than in the absence of the device.

The method as articulated in the preceding embodiment wherein the dysphagia is oropharyngeal dysphagia.

The method as articulated in one or more of the preceding embodiments wherein tongue strength is assessed by measuring tongue pressure (TP) The method as articulated in one or more preceding embodiments wherein the TRP is forward-oriented.

The method as articulated in one or more preceding embodiments wherein the TRP device is adjustable, the method further comprising adjusting the TRP during the wearing period by adjusting the position of the component back or forth in an axial direction and again securing the ends of the constraining mechanism to the respective left and right second surfaces or by removing the constraining mechanism and replacing the constraining mechanism by a second constraining mechanism having different geometry and/or orientation than the first constraining mechanism, to enhance the effect of the TRP device on tongue strength and thereby enhance the treatment of dysphagia.

The method as articulated in one or more preceding embodiments wherein the TRP device is not adjustable the method further comprising replacing the entire TRP device with one having a constraining mechanism with a different orientation and/or geometry, to enhance the effect of the TRP on tongue strength and thereby enhance the treatment of dysphagia.

The method as articulated in one or more preceding embodiments wherein the wearing period extends from 6 to 18 months.

The method as articulated in one or more preceding embodiments wherein the wearing period continues until TP reaches and maintains a normal value of at least 30 and up to 55 kPa

The method as articulated in one or more preceding embodiments wherein the component comprises an arch.

The method as articulated in one or more preceding embodiments wherein the component comprises an arch and the arch has an apex.

The method of as articulated in one or more preceding embodiments wherein the arch has a central bead.

The method as articulated in one or more preceding embodiments wherein the attachment mechanism is a circumferential retainer band.

The method as articulated in one or more preceding embodiments wherein the connector is integrated to the first and second surfaces.

### DETAILED DESCRIPTION

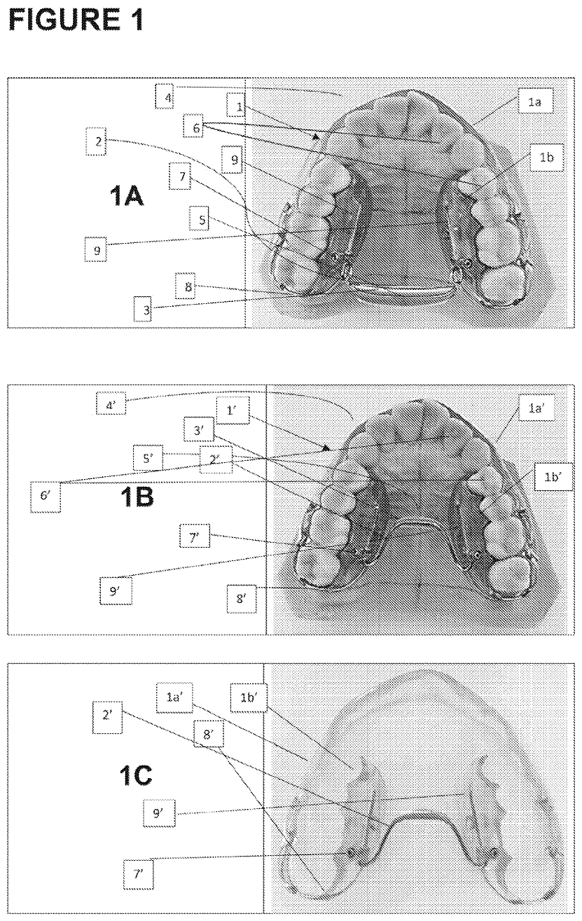

The tongue right positioner (TRP) device (exemplified in FIG. 1) is an oral device that was initially developed for orthodontic treatment [13] but has also been used to remodel the palate and more generally the oral cavity topology. As TRP aligns the tongue's position in the oral cavity, it is also used in patients with obstructive sleep apnea (OSA) [14]. The device partially inhibits the back and forward tongue thrust movements and also inhibits the tongue from exerting vertical pressure against the palate. Therefore, the present inventors hypothesized that the strength of swallowing-related muscles including muscles of the tongue might increase to compensate for the inhibited tongue movements. A previous study reported that TRP (an earlier version of the device used to generate data reported in the present disclosure) increases the strength of suprahyoid muscles, such as genioglossus (GG), geniohyoid (GH), styloglossus (SG) and stylohyoid (SH) muscles in patients who did not have dysphagia. Therefore, the inventors further hypothesized that TRP could potentially be used by choice of an arch and/or its orientation and period of treatment) to improve the strength of the tongue muscle. To the inventors' knowledge, the study disclosed herein is the first study of effects of TRP on tongue strength and function in patients with dysphagia.

### Materials and Methods

This single-arm study was conducted per the Declaration of Helsinki of 1975, revised in 2013, and was approved by the Ethics Committee of the Tokyo Medical and Dental University (D2020-023). Written informed consent was obtained from all participants.

Participants were enrolled among patients with dysphagia who visited a university dental hospital between September 2020 and September 2021. The inclusion criteria were (i) at least three of the six anterior teeth in the upper jaw remained, (ii) at least two of the six molars on both the right and left sides of the upper jaw remained. (iii) the lingual frenulum was not short, (iv) those who could handle the use of the TRP, and (v) those who agreed to join this study. The exclusion criteria were (i) inability to follow instructions, (ii) altered consciousness, and (iii) tracheostomy. Eight male patients with an average age of 58.8±12.3 years received TRP intervention. The patients were afflicted with various disorders associated with dysphagia, as detailed in Table 1 below.

The measurement variables included TP as the primary outcome and lip and tongue movements, peak nasal inspiratory flow, and changes in the tongue and suprahyoid muscle regions on ultrasonography as the secondary outcomes. Each participant was asked to use a TRP as shown in FIG. 1-B for at least 8 hours every night for 2 months. The measurement variables before and after the intervention were compared using the paired t-test and the Wilcoxon signed-rank test.

### The TRP

The type of TRP used (supplied by Tongue Lab Japan, Kyoto, Japan and commercially available through the same company or its parent. Tongue Lab Europe Ltd.), has been disclosed in detail in International (PCT) Patent Application PCT/IB2017/054130 published as WO/2018/008002 (incorporated by reference) by Tongue Lab Europe Ltd. The device was supplied by Tongue Lab Japan, Kyoto, Japan, and was provided to all participants. The particular TRP devices used in the study are custom-made for each patient, removable, retainer-based oral devices (made based on a dental and palatal impression of the patient) comprising a resin retainer 1′ and a transverse arch 2′ featuring a central bead 3′ enveloping the arch 2′ (FIGS. 1A through 1C with particular reference to FIGS. 1B and 1C), The device was positioned along the maxillary arch 4′, with the transverse arch 2′ extending between the left and right side of the upper maxilla, with the apex of the arch 5′ approximately at the mesial level of the first molars, and at the same height as the occlusal edge of upper teeth 6′ (approaching the occlusion plane, not shown). Bead 3′ is located in the middle of the arch and helps the arch oppose tongue movements. The arch 2′ can be removed by loosening screws 7′ (one on each side) and pulling it out. A replacement arch can then be used which may be of the same or a different orientation (backward as in FIG. 1A or forward as in FIGS. 1B and 1C) and dimensions (length of the base of the arch and distance of the apex from the base of the arch) and angle with respect to the occlusal plane of the wearer, not shown

In the attached Appendix, excerpted from PCT Application WO/2018/008002, exemplary arches of various shapes, angles and dimensions for inserting in the second surfaces of the TRP device have been described in FIGS. 8, 10 and 11 of the aforementioned PCT application. Further adjustments to the arch can be made by adjusting loops 8′ (one on each side). It should be noted, however, that the loops are not necessary as the arch can already be adjusted through the screws 7′ (and a corresponding nut not shown) or can be removed and replaced by an arch of different dimensions or angle. Furthermore, the present disclosure uses the described version of the TRP only as an illustration. Earlier TRP devices (described in WO/2010/015685 and WO/2012/085672) could be used and plastic, disposable TRP devices can also be used.

In general, a rearward-oriented TRP device (illustrated in FIG. 1A) is for limiting movement of a posterior zone of the tongue of the wearer while allowing an anterior zone and lateral edges of the tongue to perform movements necessary for speech and swallowing, Conversely, a forward-oriented TRP device (illustrated in FIGS. 1B and 1C) is for limiting movement of a anterior zone of the tongue of a person while allowing a posterior zone and lateral edges of the tongue (as well as the tongue's tip) to perform movements necessary for speech and swallowing, In either case, a TRP comprises an attachment mechanism, such as a retainer band (1 or 1′), adapted to attach the dental device onto teeth within the mouth of the wearer. The attachment mechanism comprises a first surface (1a or 1a′) adapted to be positioned on a vestibular side of the upper teeth of the dental arcade of the wearer and a left and a right second surfaces or returns (1b or 1b′).

Each second surface (1b or 1b′) is in opposition to the first surface and is adapted to be seated along at least one corresponding, respectively left or right, posterior tooth on the lingual side of the tooth. The second surfaces can extend along the lingual side of two or three or four posterior rear teeth of the wearer.

Each second surface is joined to the first surface by a connector (8 or 8′) which can be integrated to the first and second surfaces or can be a distinct element. In FIGS. 1A and 1B the connector is made of metal wire and is thus distinct from the resin material of the attachment mechanism (retainer).

The second surfaces are attached to a tongue-constraining mechanism which is secured, optionally removably, to the attachment mechanism and has a component (comprising what is commonly referred to as an arch) that spans between the pair of second surfaces. The arch can be considered to have an apex in the center 5 or 5′. The component has a first end attached to the first surface and a second end attached to the second surface. The component (arch) can have a forward orientation (FIG. 1B) or a rearward orientation (FIG. 1A).

The component is configured to seat at approximately an occlusal plane of the person, or at an angle thereto (the angle at the apex can be up to 30 degrees below the occlusal plane) to limit movement of the corresponding zone of the tongue (anterior or posterior) and thereby to confine the range of motion of the tongue to a more limited three-dimensional space than in the absence of the TRP device, while allowing at least the zone of the tongue that is not under constraint (anterior zone in the case of a rearward-oriented device and posterior zone in the case of a forward-oriented device) and the lateral edges of the tongue (the constraint is most pronounced in the center of the tongue where the apex of the component is) to perform movements necessary for speech and swallowing. The component inhibits the constrained portion of the tongue touching the palate.

In the present study a forward-oriented TRP device was used. It was thought that it would be easier for the elderly patients to get accustomed to it. However, a rearward-oriented device is also expected to work. In fact, there is a rationale for anticipating that a rearward-oriented device may be more effective in strengthening the tongue and treating dysphagia because the arch in such a device more directly impacts the muscles at the base of the tongue which are directly involved in passing the food bolus and swallowing.

The attachment mechanism or retainer or circumferential band is adapted to snap onto the upper teeth dental arcade of the person to releasably secure the device on the upper teeth/dental arcade.

The constraining mechanism or the attachment mechanism optionally comprises an adjustment mechanism for adjusting: (i) the angle of the component relative to the occlusal plane of the person when the device is worn and thereby controlling the extent of limitation of the movement of the tongue; or (ii) the position of the component along a longitudinal axis of the device and thereby controlling the posterior zone of the tongue wherein the constraint is to be applied; or (iii) both the angle and the position. In FIGS. 1A-1C the adjustment mechanism is a screw (7 or 7′) and nut combination.

The constraining mechanism is secured to the attachment mechanism. Alternatively, there is no adjustment mechanism and the constraining mechanism can be integral with the second surfaces, as in the case of a disposable TRP device. If present, the adjustment mechanism comprises a fastening mechanism, and the fastening mechanism preferably comprises a nut and screw or a nut and bolt combination, wherein each end of the component is slidably mounted and secured to the fastening mechanism in a longitudinally adjustable manner, forward or rearward, Adjustment of the component relative to the occlusal plane is feasible by altering the angle of the ends of the component or by removing the component and substituting another component with a different preset angle. In FIGS. 1A-1C, the ends (9 and 9′) of the component are secured to the second surface (1b and 1b′) via a screw (7 or 7′) and nut (not shown) combination.

### Conduct of the Study

During the study, participants were asked to visit the hospital three times to receive the intervention. On the first visit, dental impressions were taken to make a plaster model to be sent to a dental laboratory (Tongue Lab) to fabricate the personalized TRP device. The participants receiving their TRPs were informed about their usage on the second visit, and they were asked to wear the device for at least 8 hours a night at bedtime and while sleeping. Also, they were asked to return for a follow-up visit after 2 months to evaluate the effect of TRP use. Participants did not perform any active tongue training such as tongue exercises during the period between the second visit and the follow-up visit.

### Results

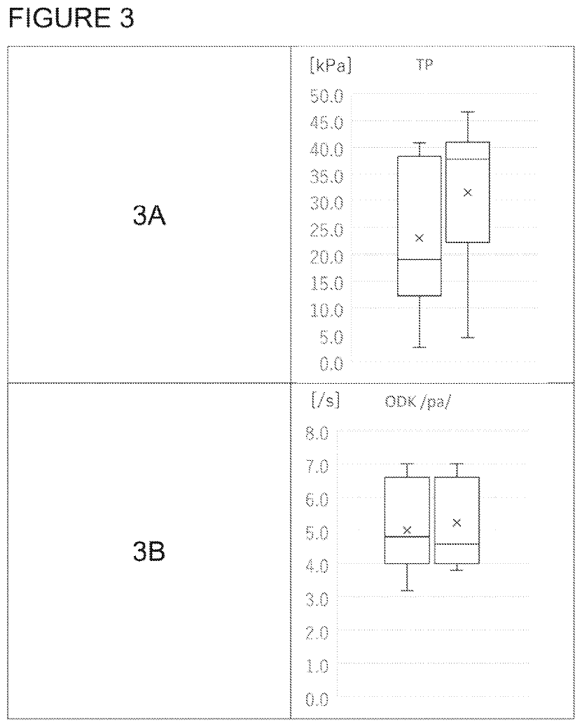

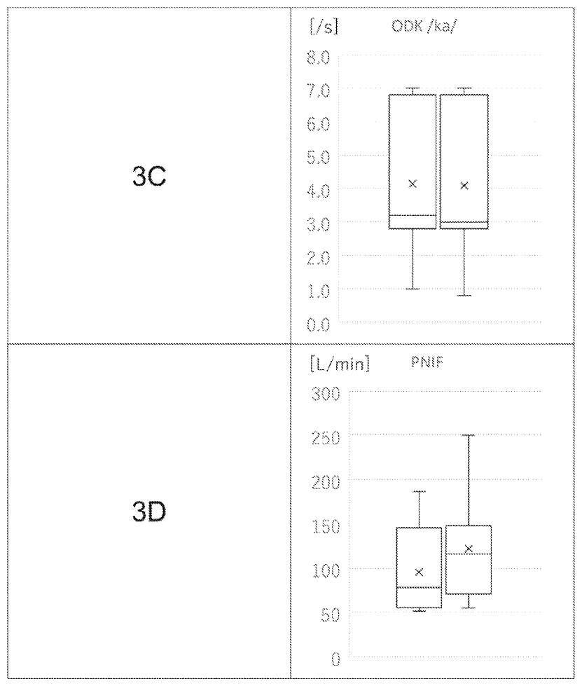

The outcome variables were assessed at the beginning of the study and at the 2-month follow-up visit. In this study, TP was the primary outcome, and the secondary outcomes were tongue and lip movement speed, peak nasal inspiratory flow (PNIF), and ultrasound assessment of swallowing-related muscles. TP after intervention (31.5±13.1 kPa) was significantly higher (p<0.034. FIG. 3A) than TP before intervention (23.0±13.4 kPa), while other measurement variables did not significantly improve (FIGS. 3B-3D). Although participants did not perform active exercises, most participants in this study observed an improved TP. Thus, the findings here show that the TRP device can greatly improve TP even after just 2 months of usage and especially considering the small number of test subjects. It should also be noted that only the forward-arched device was used in this study; the rearward-arched device was not used. We anticipate that the results with the rearward-arched device (See FIGS. 8A-C, 10A-C and 11A-C of the Appendix wherein the latter two Figures are directed to rearward-arched devices and FIG. 1A herein) may be even more effective as it targets the base of the tongue more specifically. Similarly, the other parameters measured in this preliminary study may also improve if the treatment period is longer.

### Measurements

The measurements were carried out by dentists who belong to the Department of Dysphagia Rehabilitation in a university hospital of Tokyo Medical and Dental University and are accustomed to using the measurement devices. Before the measurements, the dentists calibrated the usages of all measurement devices used in this study.

### Tongue Pressure

TP was evaluated using a TPM-01 tongue pressure measurement device (JMS Co. Ltd., Hiroshima, Japan). Accordingly, the TP values provided herein are given based on measurements with this device. Measurements with other devices may differ in number value. For example 30 to 50 kPa with TPM-01 compares to IOPI 47.5 to 69 kPa if measured with an IOPI device. See, Comparison of the Iowa Oral Performance Instrument and JMS tongue pressure measurement device https://doi.org/10.1016/j.ds.2020.06.005

Participants in the sitting position were asked to place the balloon of the device in their mouth and hold the plastic pipe with their upper and lower central incisors with their lips closed. A dentist held the probe in the correct position while recording the measurements. The participants were then asked to push the balloon with their tongue against their hard palate for 7 seconds with maximum pressure. The TP was measured three times, and the average value was recorded as described previously [15, 16].

### Tongue and Lip Movement Speed

The tongue and lip movements were evaluated using oral diadochokinesis (ODK). ODK was measured using the KENKO-KUN Handy, an oral function-measuring device (Takei Scientific Co., Ltd., Niigata, Japan). Participants were asked to pronounce a monosyllable as quickly as possible for 5 seconds. The device recorded the number of repetitions for each syllable and calculated the number of syllables produced per second. The monosyllables ‘pa,’ ‘ta,’ and ‘ka’ were used to evaluate the ability of the lips, the tip of the tongue, and the posterior region of the tongue, respectively [17, 18].

### Ultrasonographic Assessment of Swallowing-Related Muscles

The cross-sectional area of the geniohyoid muscle (CSA of the GH) and the thickness of the tongue were evaluated using an ultrasonic measuring device (SonoSite M-turbo, Fujifilm, Tokyo, Japan) in B mode [19, 20] to evaluate swallowing-related muscles. The geniohyoid (GH) muscle was selected to represent the suprahyoid muscles. Participants were asked to gently close their mouths while facing forward in the sitting position to evaluate the CSA of the GH. The probe was placed with ultrasonic gel at the midline of the mouth floor to cover the geniohyoid muscle (sagittal), and it adhered adequately to the skin without applying pressure to the tissue. The probe was placed perpendicular to the Frankfurt plane on a line connecting the first mandibular molars of the left and right, including the second premolar, to evaluate the thickness of the tongue. The probes were brought into close contact with the lower surface of the mandible covered with ultrasonic gel [20, 21]. The transverse section of the tongue was depicted at rest, with the participant facing forward in the sitting position.

The intraclass correlation coefficients (ICC) (1,1) and (2,1) were calculated to evaluate the reliability of the examiner. ICC (1,1) 0.925 and (2,1) 0.966 are used for the measurement of the CSA of the GH, and ICC (1,1) 0.936 and (2,1) 0.925 for the thickness of the tongue which revealed high reliability. ImageJ software (National Institutes of Health, Bethesda, MD. USA) was used for image processing. The CSA of the GH and tongue thickness were measured thrice and twice, respectively, and the mean values were recorded. During the analysis, the examiner was blinded to the information, including the names of the participants and whether the image was taken at baseline or at follow-up.

### Peak Nasal Inspiratory Flow (PNIF)

The PNIF was evaluated using a portable In-Check® PNIF meter (Clement Clarke International, Harlow, Essex, UK), Participants, in a seated position with their head held perpendicular to the floor, were asked to inhale with their mouths closed as firmly and quickly as possible through the mask, starting from the end of full expansion. The reliability has been established previously [22, 23], with a correlation coefficient of up to 92% [24]. The measurement was performed three times, and the average value was recorded as described previously [25].

### Other Measurements

The Barthel index (BI) and the functional oral intake scale (FOIS) scores of the participants were recorded. The BI is an index of daily living activities consisting of 10 questions, with scores between 0 and 100. A higher score was associated with a higher physical function [26]. The FOIS, a seven-point scale, was recorded to assess the oral intake level, and a higher point was associated with a higher intake level. The reliability of the BI and FOIS has been verified previously [6, 27, 28].

### Statistical Analysis

The Shapiro-Wilk test was used to test the normality of all data, after which the paired t-test and Wilcoxon signed-rank test were used for the analysis of parametric and non-parametric data, respectively, using the Japanese version of SPSS for Windows (version 25 J; IBM Japan, Ltd., Tokyo, Japan). Differences with a corrected p-value<0.05 were considered significant. A post hoc analysis was performed to calculate the effect size (ES) of each variable using G*Power 3.1 (Kiel University, Kiel, Germany). ES was defined as large for r>0.5, medium for 0.3<r<0.5, small for 0.1<r<0.3, and without effect for r<0.1.

### Results

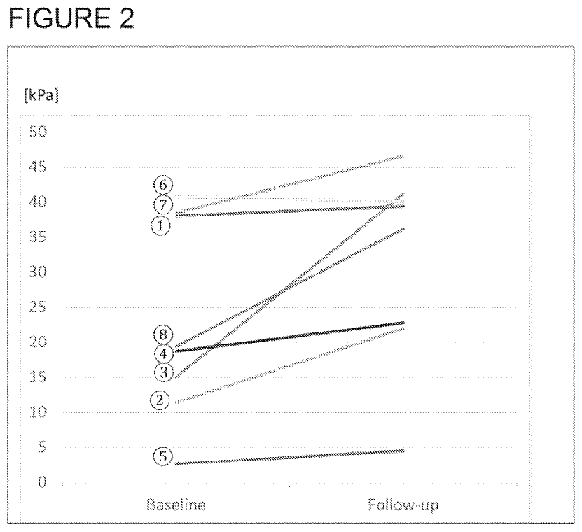

Eight participants (all men, average age of 58.8±12.3 years) were enrolled in this study. Table 1 shows the characteristics of the participants. Dysphagia-causing diseases included neuromuscular diseases (n=3) and cerebrovascular diseases (n=2). The participants' median BI and FOIS scores were 85 (25-100) and 6.5 (2-7), respectively. Table 2 shows the differences in TP, ODK, PNIF, and ultrasonographic assessment of swallowing-related muscles before and after TRP use. There was a significant improvement in TP (p=0.034, r=0.84). The ES of each measurement item was also calculated. Of the eight participants, the ODK of one participant with cerebral infarction could not be evaluated owing to expressive aphasia after cerebral infarction. There were no other missing data. FIG. 2 shows a comparison of TP between the baseline and follow-up values of each participant. Of the eight participants, seven showed improvements in TP. FIG. 3A-D shows box plots of the measurement parameters (TP, ODK/pa/, ODK/ka/and PNIF respectively) with significant improvement between the baseline TP value and the TP value at evaluation. The remaining parameters showed no improvement in this study.

In FIG. 3, The left and right ( ) box plots in each measurement item reveal baseline and follow-up values, respectively. The top and bottom of the vertical line show the maximum and minimum values, respectively, and the box in the middle illustrates the interquartile range. The horizontal line in the middle of the light grey box shows the median value, and the cross mark shows the mean value. TP, tongue pressure; ODK, oral diadochokinesis; PNIF, peak nasal inspiratory flow.

The TRP was employed for patients with dysphagia for the first time, showing that TP increased after 2-months' use. Interestingly, TP increased even though three participants had progressive disease (amyotrophic lateral sclerosis, spinocerebellar degeneration, progressive supranuclear palsy). Thus, the device can serve as a valuable tool for functional rehabilitation of the tongue in patients with oropharyngeal dysphagia even if due to underlying degenerative disease.

A decrease in TP was observed in only one among the eight participants. However, the TP of this participant at baseline was 40.8 kPa, which was not that low despite the presence of dysphagia, and the decrease was only 0.8 kPa. Thus, TRP may not have been very useful in this case, or the treatment interval was too short. However, based on the data. TRP can be said to be suitable for dysphagia associated with low tongue strength.

There have been no reports on an association between TRP and TP improvement and consequently no mechanism for such has been proposed. Without wishing to be bound by theory, a possible explanation might be the lingual-hypoglossal reflex. When filiform papillae on the tongue are mechanically stimulated, the tongue's tip curves upward, the tongue hollows from side to side with the upward curvature of the lateral edges, and the posterior part is depressed [29, 30]. TRP may cause this reflex and could enhance the strength of the tongue's anterior intrinsic and extrinsic muscles, especially the genioglossus (GG), which play a role in protrusion and retrusion. At least tongue protrusion has been reported to be involved in swallowing. The improved tongue muscles could be responsible for increased TP. The relationship between the tongue protrusion strength in arousal state and upper airway patency has been previously reported [31]. TP evaluation suggests that TRP improves the GG muscle strength, contributing to tongue protrusion. GG are pharynx dilator muscles, this could explain why TRP has been used to manage OSA [14].

Ultrasonographic evaluation revealed that the CSA of the GH did not improve in this study (p=0.484, r=0.25). Although an association between TP and the geniohyoid muscle has been reported [13], the mechanism of increased TP with TRP use could be due to the enhanced GG and SG tongue muscles rather than the suprahyoid muscles. It remains unclear whether TRP improved the GH muscle function in this study due to the small number of patients and the short treatment interval; therefore, additional research is required to clarify the effectiveness of TRP on the GH muscle function in a larger and longer study. The thickness of the tongue did not increase (p=0.588, r=0.21) despite the improvement of TP. Tongue muscle strength increases without muscle hypertrophy at the beginning of muscular exercises due to neural adaptation [32]. The improvement in TP in this study can be attributed to this phenomenon and a longer study duration, optionally and even preferably with the use of or a switch to a TRP device with a backward arch as in FIG. 18 could likely lead to increased muscle strength as well.

The ODK and PNIF were also measured. The ‘Pa’ of ODK (r=0.45) and PNIF (r=0.48) during the follow-up visit tended to increase, but when compared with that of baseline, there were no significant differences. There may be a significant difference between baseline and follow-up values in future studies with a larger sample size and/or longer study duration. In this study, cases of some patients with progressive disease should have been adjusted or homogenized. Although the improvement was not significant, the PNIF scores increased. This finding is consistent with previous studies, although the previous studies did not involve dysphagia nor measured tongue pressure [33]. Similarly, the ‘Pa’ of ODK also increased. However, the reason behind this finding remains unclear, A possible explanation might be the reduction in mouth breathing caused by increased nasal patency and related PNIF. Previously, adult mouth breathers had reduced nasal patency and had lower PNIF scores than nasal breathers [34]. In particular, mouth breathing is associated with weak lip muscle strength [35]. Accordingly, TRP improved lip closure may have been reflected in the ‘pa’ of ODK and PNIF. In future studies, the lip-closure strength should also be measured.

In contrast, the ‘ta’ and ‘ka’ of ODK decreased slightly. In the ‘ta’ of the ODK, the score of four participants, including the three participants with progressive neuromuscular diseases, increased while that of the other two decreased. The ‘ka’ scores of two participants decreased, while no change was observed in that of the remaining participants. The improvement in the ‘ta’ could be explained by enhanced genioglossal muscle strength, similar to the improvement in TP. However, the ‘ka’ did not change or decrease. Of the two participants whose ‘ka’ decreased, one had progressive disease, and the other had a history of cerebral infarction. The ‘ta’ and ‘ka’ of ODK reflect the anterior and posterior movements of the tongue, respectively. In both cases, the TRP could maintain the movement of the anterior tongue despite the presence of progressive disease.

This study had some limitations. First, only eight participants were included. Therefore, more studies are required with a larger number of male and female participants to clarify the reasons behind the effectiveness of TRP use in improving oral function. Second, although TP increased, the ultrasound assessment showed no improvement, suggesting that the muscle characteristics did not change after wearing TRP for two months. Third, the increase in TP may be due to the lingual-hypoglossal reflex. However, it was difficult to prove that the reflex had actually occurred. Most important, the duration of the wearing period was significantly shorter than the optimal wearing period for other TRP uses (6 to 18 months), so we anticipate that benefits may increase with longer use and that muscle strength increase may even be observed after longer TRP use and optionally with use of or a switch to a device with a backward arch.

This is the first study to consider the swallowing function of TRP users with dysphagia. A randomized controlled trial should be conducted to elucidate the effectiveness of TRP in dysphagia and its accompanying abnormal values of the parameters measured in this study. In future studies. Considering that the ES of TP is 0.84 in this study, 48 participants, including 24 with TRP and placebo oral device (α=0.05, power=0.8), will be recruited. The length of treatment may be prolonged to at least 4 months and preferably 6 months to 1 year, as in the case of other indications of the TRP benefits continue to increase. Furthermore, in future studies, swallowing evaluations with videofluorography or videoendoscopy should be conducted.

Numerous exercises have been suggested to improve TP [12, 38, 39]. However, most require patients to follow instructions. In contrast, although participants did not perform active exercises, most participants in this study observed an improvement in TP.

TRP is a noninvasive and removable oral device worn entirely inside the mouth that could be useful for patients with dysphagia who cannot perform or are unwilling to perform active exercises and sustain an active exercise regime. Additionally, caregiver assistance is required, if at all, only during device insertion and removal. Therefore, TRP can be used even in situations with limited assistance. No adverse events were reported in this study.

### Tables

TABLE 1
Characteristics of participants

| | | | | | | | TP at | TP at | |
|---|---|---|---|---|---|---|---|---|---|
| No. | Age | Sex | Weight | BMI | BI | FOIS | baseline | follow-up | Past history |
| 1 | 56 | M | 106 | 35.8 | 100 | 7 | 38.0 | 39.4 | Cerebral tumor |
| 2 | 72 | M | 44 | 17.2 | 35 | 3 | 11.3 | 21.9 | Progressive |
| | | | | | | | | | supranuclear palsy |
| 3 | 49 | M | 78 | 26.7 | 70 | 6 | 15.0 | 41.1 | Spinocerebellar |
| | | | | | | | | | degeneration |
| 4 | 62 | M | 65 | 26.4 | 45 | 5 | 18.7 | 22.7 | Amyotrophic lateral |
| | | | | | | | | | sclerosis |
| 5 | 62 | M | 68 | 24.1 | 25 | 2 | 2.6 | 4.5 | Cerebral infarction |
| 6 | 67 | M | 59 | 22.5 | 100 | 7 | 40.8 | 40.0 | Subjective symptoms of |
| | | | | | | | | | swallowing difficulty |
| 7 | 70 | M | 71 | 28.4 | 100 | 7 | 38.4 | 46.5 | Wallenberg syndrome |
| 8 | 32 | M | 71 | 21.2 | 100 | 7 | 19.4 | 36.1 | Subjective symptoms of |
| | | | | | | | | | swallowing difficulty |

BMI, body mass index; BI, Barthel index; FOIS, functional oral intake scale; TP, tongue pressure.

TABLE 2
Comparison of each measurement item between baseline and follow-up values

| | | Baseline | | Follow-up | | | |
|---|---|---|---|---|---|---|---|
| | | | Median | | Median | | |
| Measurements | | Mean ± | (Interquartile | Mean ± | (Interquartile | | Effect |
| (n = 8) | | S.D. | range) | S.D. | range) | P-value | size (r) |
| TP^{†} | | 23.0 ± | 19.0 | 31.5 ± | 37.8 | 0.034* | 0.84 |
| | | 13.4 | (14.1-38.1) | 13.1 | (22.5-40.3) | | |
| ODK | /pa/^{‡} | 5.0 ± | 4.8 | 5.2 ± | 4.6 | 0.285 | 0.45 |
| | | 1.3 | (4.0-6.0) | 1.3 | (4.0-6.6) | | |
| | /ta/^{†} | 5.0 ± | 4.8 | 4.9 ± | 4.6 | 0.668 | 0.27 |
| | | 1.5 | (3.6-6.6) | 1.4 | (3.9-5.7) | | |
| | /ka/^{†} | 4.1 ± | 3.2 | 4.1 ± | 3.0 | 0.172 | 0.53 |
| | | 2.1 | (2.8-6.1) | 2.1 | (2.8-6.1) | | |
| PNIF^{†} | | 95.8 ± | 78.3 | 122.1 ± | 115.8 | 0.197 | 0.48 |
| | | 48.4 | (55.8-114.2) | 58.6 | (73.8-147.1) | | |
| Ultrasonographic | CSA of | 174.9 ± | 178.8 | 181.2 ± | 188.0 | 0.484 | 0.25 |
| assessment | GH^{‡} | 14.7 | (175.1-182.6) | 24.0 | (174.4-194.5) | | |
| | Thickness | 30.5 ± | 30.4 | 29.8 ± | 30.6 | 0.588 | 0.21 |
| | of tongue^{†} | 3.9 | (28.5-33.7) | 3.4 | (28.2-33.7) | | |

S.D., standard deviation; TP, tongue pressure; ODK, oral diadochokinesis; PNIF, peak nasal inspiratory flow; CSA of GH, cross-sectional area of the geniohyoid muscle.
*Statistically significant (p < 0.05).
^{†}Paired t-test was performed for TP, /ta/, and /ka/ of ODK, PNIF, and the thickness of the tongue in ultrasonographic measurement.
^{‡}Wilcoxon signed-rank test was performed for /pa/ of ODK and CSA of GH in ultrasonographic measurement.
All cited references are incorporated by reference, each in its entirety for all purposes.

### REFERENCES

1. Aoki Y, Kabuto S, Ozeki Y, Tanaka T, Ota K (2015) The effect of tongue pressure strengthening exercise for dysphagic patients. Jpn J Compr Rehabil Sci 6:129-136. https://doi.org/10.11336/jjcrs.8.129
2. Hara K, Tohara H, Kenichiro K, Yamaguchi K, Ariya C, Yoshimi K, Nakane A. Minakuchi S (2019) Association between tongue muscle strength and masticatory muscle strength. J Oral Rehabil 46:134-139. https://doi.org/10.1111/joorr.1237
3. Nakamori M, Hosomi N, Ishikawa K, Imamura E, Shishido T, Ohshita T, Yoshikawa M, Tsuga K, Wakabayashi S, Maruyama H, Matsumoto M (2016) Prediction of pneumonia in acute stroke patients using tongue pressure measurements. PLOS ONE 11:e0165837. https://doi.org/10.1371/journal.pone.0165837
4. Umemoto G. Tsuboi Y, Kitashima A, Furuya H, Kikuta T (2011) Impaired food transportation in Parkinson's disease related to lingual bradykinesia. Dysphagia 26:250-255. https://doi.org/10.1007/s00455-010-9296-y
5. Minagi Y, Ono T, Hori K, Fujiwara S, Tokuda Y, Murakami K, Maeda Y, Sakoda S, Yokoe M, Mihara M, Mochizuki H (2018) Relationships between dysphagia and tongue pressure during swallowing in Parkinson's disease patients. J Oral Rehabil 45:459-466. https://doi.org/10.1111/joor.12626
6. Maeda K, Akagì J (2015) Decreased tongue pressure is associated with sarcopenia and sarcopenic dysphagia in the elderly. Dysphagia 30:80-87. https://doi.org/10.1007/s00455-014-9577-v
7. Namasivayam A M, Steele C M, Keller H (2016) The effect of tongue strength on meal consumption in long term care. Clin Nutr 35:1078-1083. https://doi.org/10.1016/j.clnu.2015.08.001
8. Nakamori M. Ishikawa K, Imamura E, Yamamoto H, Kimura K, Ayukawa T. Mizoue T, Wakabayashi S (2021) Relationship between tongue pressure and dysphagia diet in patients with acute stroke. PLOS ONE 16: e0252837. https://doi.org/10.1371/journal.pome.025837
9. Ono T. Kumakura I, Arimoto M. Hori K, Dong J. Iwata H, Nokubi T. Tsuga K, Akagawa Y (2007) Influence of bite force and tongue pressure on oropharyngeal residue in the elderly. Gerodontology 24:143-150. https://doi.org/10.1111/j.1741-2358.2007.00172.x
10. Robbins J, Gangnon R E, Theis S M, Kays S A, Hewitt A L, Hind J A (2005) The effects of lingual exercise on swallowing in older adults. J Am Geriatr Soc 53:1483-1489. https://doi.org/10.1111/j.1532-5415.2005.53467.x
11. Fujiu-Kurachi M, Fujiwara S, Tamine K I, Kondo J. Minagi Y, Maeda Y, Hori K, Ono T (2014) Tongue pressure generation during tongue-hold swallows in young healthy adults measured with different tongue positions. Dysphagia 29:17-24. https://doi.org/10.1007/s00455-013-947-z
12. Namiki C. Hara K, Tohara H. Kobayashi K, Chantaramanee A, Nakagawa K, Saitou T, Yamaguchi K, Yoshimi K, Nakane A, Minakuchi S (2019) Tongue-pressure resistance training improves tongue and suprahyoid muscle functions simultaneously. Clin Interv Aging 14:601-608. https://doi.org/10.2147/CIA.S194808
13. Mauclaire C, Vanpoulle F, Saint-Georges-Chaumet Y (2015) Saint-Georges-Chaumet Y. Physiological correction of lingual dysfunction with the ‘Tongue Right Positioner’: Beneficial effects on the upper airways. Int Orthod 13:370-389. https://doi.org/10.1016/j.ortho.2015.06.007
14. Wullman P, Belattar A, Coulson S, Vanpoulle F, Mauclaire C, Saint-Georges-Chaumet Y (2019) Effet de la rééducation linguale avec Tongue Right Positioner sur le SAOS. Med Sommeil 16:43 (in French). https://www.researchgate.net/publication/337632031_Interets_de_la_reeducation_linguale_avec_le_dispositif_TRP_pour_Je_traitement_des_troubles_respiratoires_obstructifs_du_som meil. https://doi.org/10.1016/j.mscm.2019.01.065. See also International PCT Patent Publication WO/2018/008002.
15. Utanohara Y, Hayashi R, Yoshikawa M, Yoshida M, Tsuga K, Akagawa Y (2008) Standard values of maximum tongue pressure taken using newly developed disposable tongue pressure measurement device. Dysphagia 23:286-290. https://doi.org/10.1007/s00455-007-9142-z
16. Shimizu A, Fujishima I. Maeda K, Wakabayashi H, Nishioka S, Ohno T, Nomoto A, Shigematsu T, Kayashita J; Japanese Working Group on Sarcopenic Dysphagia (2021) Effect of low tongue pressure on nutritional status and improvement of swallowing function in sarcopenic dysphagia. Nutrition 90:111295. https://doi.org/10.1016/j.nut.2021.111295
17. Ito K, Yoshihara A, Takano N, Ishigami K, Seida Y. Inoue M, Kitahara M, Miyazaki H (2009) A comparison of methods for the measurement of oral diadochokinesis. Ronen Shika Igaku (Jpn J Gerodontol) 24:48-54 (in Japanese). https://doi.org/10.11259/jsg.24.48
18. Ziegler W (2002) Task-related factors in oral motor control: Speech and oral diadochokinesis in dysarthria and apraxia of speech. Brain Lang 80:556-575. https://doi.org/10.1006/brin.2001.2614
19. Yabunaka K, Konishi H, Nakagami G, Sanada H, Iizaka S, Sanada S, Ohue M (2012) Ultrasonographic evaluation of geniohyoid muscle movement during swallowing: A study on healthy adults of various ages. Radiol Phys Technol 5:34-39. https://doi.org/10.1007/s12194-011-0132-3
20. Ogawa N, Mori T, Fujishima I, Wakabayashi H, Itoda M, Kunieda K, Shigematsu T, Nishioka S, Tohara H, Yamada M. Ogawa S (2018) Ultrasonography to measure swallowing muscle mass and quality in older patients with sarcopenic dysphagia. J Am Med Dir Assoc 19:516-522. https://doi.org/10.1016/j.jarnda.2017.11.007
21. Yoshimi K, Hara K, Tohara H, Nakane A, Nakagawa K, Yamaguchi K, Kurosawa Y, Yoshida S, Ariya C, Minakuchi S (2018) Relationship between swallowing muscles and trunk muscle mass in healthy elderly individuals: A cross-sectional study. Arch Gerontol Geriatr 79:21-26. https://doi.org/10.1016/j.archer2018.07.018
22. Cho S I, Hauser R, Christiani D C (1997) Reproducibility of nasal peak inspiratory flow among healthy adults: Assessment of epidemiologic utility. Chest 112:1547-1553. https://doi.org/10.1378/chest.112.6.1547
23. Teixeira R U F, Zappelini C E M, Alves F S, da Costa E A (2011) Peak nasal inspiratory flow evaluation as an objective method of measuring nasal airflow. Braz J Otorhinolaryngol 77:473-480. https://doi.org/10.1590/S1808-86942011000400011
24. Starling-Schwanz R, Peake H L, Salome C M, Toelle B G, Ng K W, Marks G B, Lean M L, Rimmer S J (2005) Repeatability of peak nasal inspiratory flow measurements and utility for assessing the severity of rhinitis. Allergy 60:795-800. https://doi.org/10.1111/j.1398-995.2005.00779.x
25. Sawa A, Suzuki H, Niwa H, Oguchi S, Yagi T, Iwata Y, Makiyama Y, Chow C M, Komiyama O (2020) Assessment of screening for nasal obstruction among sleep dentistry outpatients with obstructive sleep apnea. Dent J (Basel) 8:119. https://doi.org/10.3390/dj8040119
26. Mahoney F I, Barthel D W (1965) Functional evaluation: The Barthel index. Md State Med J 14:61-65
27. Sainsbury A, Seebass G, Bansal A, Young J B (2005) Reliability of the Barthel index when used with older people. Age Ageing 34:228-232. https://doi.org/10.1093/ageing/afi063
28. Crary M A, Mann G D C, Groher M E (2005) Initial psychometric assessment of a functional oral intake scale for dysphagia in stroke patients. Arch Phys Med Rehabil 86:1516-1520. https://doi.org/10.1016/j.aprnr.2004.11.049
29. John R R, Chandrasekaran B, Murugadoss P (2019) Lingual hypoglossal reflex: An unusual reflex of head and neck. J Maxillofac Oral Surg 18:366-370. https://doi.org/10.1007/s12663-018-1157-5
30. Miller A J (2002) Oral and pharyngeal reflexes in the mammalian nervous system: Their diverse range in complexity and the pivotal role of the tongue. Crit Rev Oral Biol Med 13:409-425. https://doi.org/10.1177/154411130201300505
31. Kanezaki M, Ogawa T, Izumi T (2015) Tongue protrusion strength in arousal state is predictive of the airway patency in obstructive sleep apnea. Tohoku J Exp Med 236:241-245. https://doi.org/10.1620/tjem.236.241
32. Cullins M J, Krekeler B N, Connor N P (2018) Differential impact of tongue exercise on intrinsic lingual muscles. Laryngoscope 128:2245-2251. https://doi.org/10.1002/iary.27044
33. Mauclaire C, Vanpoulle F, Belattar A (2017) Saint-Georges-Chaumet Y. Importance of the lingual reeducation by the tongue right positioner on the upper airways permeability in young orthodontic patients. Sleep Med 24:161-165
34. Trevisan M E, Bellinaso J H, Pacheco Ade Ade B, Augé L B, Silva A M, Corrêa E C (2015) Respiratory mode, nasal patency and palatine dimensions. CoDAS. Sociedade Brasileira de Fonoaudiologia 2015. Codas 27:201-206 [sin Protuguese]. https://doi.org/10.1590/2317-1782/20162014177
35. Ismail A M A (2021) Oral rehabilitation using the lip muscle trainer: A narrative review. PTJ 11:139-144. https://doi.org/10.32598/pti.11.3.486.1
36. Horibe Y, Matsuo K, Ikebe K. Minakuchi S, Sato Y, Sakurai K, Ueda T (2022) Relationship between two pressure-sensitive films for testing reduced occlusal force in diagnostic criteria for oral hypofunction. Gerodontology 39:3-9. https://doi.org/10.1111/ger.12538
37. Yanagida R. Hara K, Iida T, Tohara T, Tamada Y. Minakuchi S, Namiki C. Okumura T, Tohara H (2022) Jaw-opening force as a useful index for dysphagia: A cross-sectional and multi-institutional study. Gerontology:1-8. https://doi.org/10.1159/000521392
38. Park T, Kim Y (2016) Effects of tongue pressing effortful swallow in older healthy individuals. Arch Gerontol Geriatr 66:127-133. https://doi.org/10.1016/j.archger.2016.05.009
39. Oh J C (2021) Effects of resistive jaw-opening exercise with elastic resistance bands on suprahyoid muscle activation and tongue strength in the elderly: A pilot study. Folia Phoniatr Logop 73:376-383. https://doi.org/10.1159/000509441
40. Sakamoto, Y. et al. Effect of decreased tongue pressure on dysphagia and survival rate in elderly people requiring long-term car. (2022) Journal of Dental Sciences 17: 856-862
41. Sakai, K. et al. Association of Oral Func and Dysphagia with Frailty and Sarcopenia in Community-Dwelling Older Adults: A Systematic Review and Meta-Analysis. Cells (2022) 11, 2199. https://doi.org/10.3390/cells11142199.
42. Milazzo, M. et al, Tongue Rehabilitation Device for Dysphagic Patients. Sensors (2019) 19, 4657: doi:10.3390/s19214657

FIGS. 8A-C show exemplary constraining mechanisms having a forward orientation and horizontal loops in accordance with one or more embodiments. FIG. 8A shows exemplary lengths of a forward-oriented short constraining mechanism (FwS) and a forward-oriented long constraining mechanism (FwL) in accordance with one or more embodiments. FIG. 8B shows a front perspective view of the exemplary forward-oriented constraining mechanism with horizontal loops, and FIG. 8C shows a rear view of the exemplary forward-oriented constraining mechanism in accordance with one or more embodiments;

FIGS. 10A-C show exemplary constraining mechanisms having a backward (rearward) orientation in accordance with one or more embodiments. FIG. 10A shows exemplary lengths of a backward-oriented short constraining mechanism (BwS) and a backward-oriented long constraining mechanism (BwL) in accordance with one or more embodiments. FIG. 10B shows a front view of the exemplary backward-oriented constraining mechanism, and FIG. 10C shows a top view of the exemplary backward-oriented constraining mechanism in accordance with one or more embodiments;

FIGS. 11A-C show exemplary constraining mechanisms having a backward orientation and horizontal loops in accordance with one or more embodiments. FIG. 11A shows exemplary lengths of a backward-oriented short constraining mechanism (BwS) and component thereof and a backward-oriented long constraining mechanism (BwL) and component thereof in accordance with one or more embodiments. FIG. 11B shows a front view of the exemplary backward-oriented constraining mechanism with horizontal loops, and FIG. 11C shows a top view of the exemplary backward-oriented constraining mechanism in accordance with one or more embodiments.

1. A method for treating dysphagia comprising:
fitting a patient afflicted with dysphagia with a TRP oral retainer-based device for wearing at night starting at about bedtime and during sleep, preferably for at least 8 hours;
providing instructions to the patient to wear the device nightly for a period of time from about 2 to about 18 months or longer (the wearing period) as indicated based on strength of the patient's tongue; monitoring the patient's tongue pressure at one or more time points during the wearing period,
wherein an increase in tongue strength compared to baseline, or compared to a prior monitoring time point during the wearing period, indicates that the patient receives a benefit from wearing the device or continues to receive a benefit from wearing the device and the patient's dysphagia is thereby treated; and
wherein the TRP device is for limiting movement of a posterior or anterior zone of the tongue and stimulating the tongue, while allowing respectively an anterior or posterior zone and lateral edges of the tongue to perform movements necessary for speech and swallowing and comprises:
an attachment mechanism adapted to releasably attach the TRP device teeth of the patient, the attachment mechanism comprising
a first surface adapted to be positioned on a vestibular side of upper teeth of the dental arcade of the patient and
a pair of second surfaces, comprising a left second surface and a right second surface, each second surface in opposition to the first surface and adapted to be seated along at least one and up to four posterior teeth of the wearer on the lingual side of the posterior teeth and
a connector for joining a left end of the first surface with the left second surface and a connector for joining a right end of the first surface with the right second surface
a tongue-constraining mechanism having
a first and second end wherein each end of the constraining mechanism is optionally removably secured to a corresponding second surface of the attachment mechanism and
a component transverse to and spanning between the first and second surfaces and having a forward or rearward orientation, the component being configured to sit at the occlusal plane of the patient or at an angle thereto the angle being within the range from +0 to −30 degrees lower than the occlusal plane and to limit movement of the corresponding zone of the tongue thereby confining the range of motion of the tongue to a more limited three-dimensional space within the mouth of the patient than in the absence of the device.

2. The method of claim 1 wherein the dysphagia is oropharyngeal dysphagia.

3. The method of claim 1 wherein tongue strength is assessed by measuring tongue pressure (TP)

4. The method of claim 1 wherein the TRP is forward-oriented.

5. The method of claim 1 wherein the TRP device is adjustable, the method further comprising adjusting the TRP during the wearing period by adjusting the position of the component back or forth in an axial direction and again securing the ends of the constraining mechanism to the respective left and right second surfaces or by removing the constraining mechanism and replacing the constraining mechanism by a second constraining mechanism having different geometry and/or orientation than the first constraining mechanism, to enhance the effect of the TRP device on tongue strength and thereby enhance the treatment of dysphagia.

6. The method of claim 1 wherein the TRP device is not adjustable the method further comprising replacing the entire TRP device with one having a constraining mechanism with a different orientation and/or geometry, to enhance the effect of the TRP on tongue strength and thereby enhance the treatment of dysphagia.

7. The method of claim 1, wherein the wearing period extends from 6 to 18 months.

8. The method of claim 1 wherein the wearing period continues until TP reaches and maintains a normal value of at least 30 and up to 55 kPa.

9. The method of claim 1 wherein the component is an arch.

10. The method of claim 8 wherein the arch has an apex.

11. The method of claim 9 wherein the arch has a central bead.

12. The method of claim 1 wherein the attachment mechanism is a circumferential retainer band.

13. The method of claim 1 wherein the connector is integrated to the first and second surfaces.
